(19) United States
(12) Patent Application Publication
(10) Pub. No.: US 2003/0225332 A1
(43) Pub. Date: Dec. 4, 2003

(54) Ultrasonic therapeutic apparatus

(75) Inventors: Mitsumasa Okada, Tokyo (JP); Satoshi Honma, Tokyo (JP)

Correspondence Address: Thomas Spinelli, Scully, Scott, Murphy & Presser, 400 Garden City Plaza, Garden City, NY, 11530, US

(73) Assignee: OLYMPUS OPTICAL CO., LTD., Tokyo (JP)

(21) Appl. No.: 10/445,581

(22) Filed: May 27, 2003

(30) Foreign Application Priority Data
May 31, 2002 (JP) 2002-160557

(51) Int. Cl. A61B 8/00

(52) U.S. Cl. 600/439

(57) ABSTRACT

An ultrasonic therapeutic apparatus is disclosed which includes an endoscope having an inserting section to be inserted into an object, an ultrasonic vibrator for generating ultrasonic vibration, a vibration transmitting section for transmitting the ultrasonic vibration generated by the ultrasonic vibrator to the object, and a holding member for holding the ultrasonic vibrator and the vibration transmitting section in a tip portion of the inserting section of the endoscope. This construction does not need the process of positioning the ultrasonic therapeutic apparatus with respect to the endoscope. An example of the vibration transmitting section is a probe (horn).

### CROSS-REFERENCE TO RELATED APPLICATION

This application is based upon and claims the benefit of priority from prior Japanese Patent Application No.2002-160557, filed May 31, 2002, the entire contents of which is incorporated herein by reference.

### BACKGROUND OF THE INVENTION

1. Field of the Invention

The present invention relates to an ultrasonic therapeutic apparatus and, more particularly, to an ultrasonic therapeutic apparatus used under endoscopic observation.

2. Description of the Related Art

An ultrasonic therapeutic apparatus which is used under endoscopic observation has heretofore been known.

Each of JP-A-1987-299251 and JP-A-2001-37768 discloses an ultrasonic therapeutic apparatus including an ultrasonic therapeutic probe fitted in a hand piece containing an ultrasonic vibrator. Specifically, in either of the disclosed arts, the hand piece of the ultrasonic therapeutic apparatus is fitted to an operating section of an endoscope, and the ultrasonic therapeutic probe is inserted into a channel of the endoscope to perform ultrasonic treatment under endoscopic observation. JP-A-1987-299251 discloses a combination of an ultrasonic therapeutic apparatus and a flexible endoscope, while JP-A-2001-37768 discloses a combination of an ultrasonic therapeutic apparatus and a rigid endoscope.

In these related arts, the process of inserting an elongated ultrasonic probe into a channel of an endoscope takes place before or during an operation. However, accurate and reliable insertion of the ultrasonic probe into the endoscope is a difficult process. It is preferable to dispose the ultrasonic probe at a position where part of the ultrasonic probe becomes visible in the field of view of the endoscope so that treatments can be performed reliably and safely, but users often feel stress from the process of accurately disposing the ultrasonic probe at such a position.

### BRIEF SUMMARY OF THE INVENTION

An ultrasonic therapeutic apparatus according to the invention includes an endoscope having an inserting section to be inserted into an object, an ultrasonic vibrator for generating ultrasonic vibration, a vibration transmitting section for transmitting the ultrasonic vibration generated by the ultrasonic vibrator to the object, and a holding member for holding the ultrasonic vibrator and the vibration transmitting section in a distal tip portion of the inserting section of the endoscope.

Accordingly, since the ultrasonic vibrator and the vibration transmitting section are held in the distal tip portion of the inserting section of the endoscope, there is no need for the process of inserting the ultrasonic therapeutic apparatus into the endoscope and positioning the ultrasonic therapeutic apparatus with respect to the endoscope before and during an operation.

An example of the vibration transmitting section is a probe (horn). For good operability, a suction passage may be provided in the probe. Preferably, the probe and the ultrasonic vibrator are removably disposed so that an appropriate probe can be selected according to the contents of an operation to be applied. In this case, the shape of the probe may be a cylinder, a cone, a hook, a spatula or the like.

For good operability, the distal tip portion of the probe may be positioned in the field of view of the endoscope. The probe may be constructed to be movable back and forth, rotatable and tiltable so that the probe can be easily positioned at an appropriate location.

In the case of a tiltable probe, a jaw may be provided in the distal tip portion of the endoscope so that an object can be clamped between the probe and the jaw.

### BRIEF DESCRIPTION OF THE SEVERAL VIEWS OF THE DRAWINGS

These and other features, aspects, and advantages of the apparatus and methods of the present invention will become better understood with regard to the following description, appended claims, and accompanying drawings where:

FIG. 1 is a view showing an ultrasonic therapeutic apparatus according to a first embodiment of the invention;

FIG. 2A is an enlarged cross-sectional view of a distal tip portion of the ultrasonic therapeutic apparatus shown in FIG. 1;

FIG. 2B is an enlarged front view of the distal tip portion of the ultrasonic therapeutic apparatus shown in FIG. 1;

FIG. 3 is a cross-sectional view showing the distal tip-end side of an ultrasonic therapeutic apparatus body according to a second embodiment of the invention;

FIG. 4 is a perspective view showing the distal tip-end side of an ultrasonic therapeutic apparatus body according to a variation of the second embodiment;

FIG. 5 is a cross-sectional view showing the distal tip-end side of an ultrasonic therapeutic apparatus body according to a third embodiment of the invention;

FIG. 6 is a cross-sectional view showing the distal tip-end side of an ultrasonic therapeutic apparatus body according to a fourth embodiment of the invention;

FIG. 7 is a cross-sectional view showing the distal tip-end side of an ultrasonic therapeutic apparatus body according to a fifth embodiment of the invention;

FIG. 8 is a cross-sectional view showing the distal tip-end side of an ultrasonic therapeutic apparatus body according to a sixth embodiment of the invention;

FIG. 9 is a perspective view showing the distal tip-end side of the ultrasonic therapeutic apparatus body shown in FIG. 8;

FIG. 10 is a view showing a specific example of the observing field of view of the ultrasonic therapeutic apparatus body shown in FIG. 8; and

FIG. 11 is a perspective view showing the distal tip-end side of an ultrasonic therapeutic apparatus body according to a seventh embodiment of the invention.

### DETAILED DESCRIPTION OF THE INVENTION

Preferred embodiments of the invention will be described below with reference to the accompanying drawings.

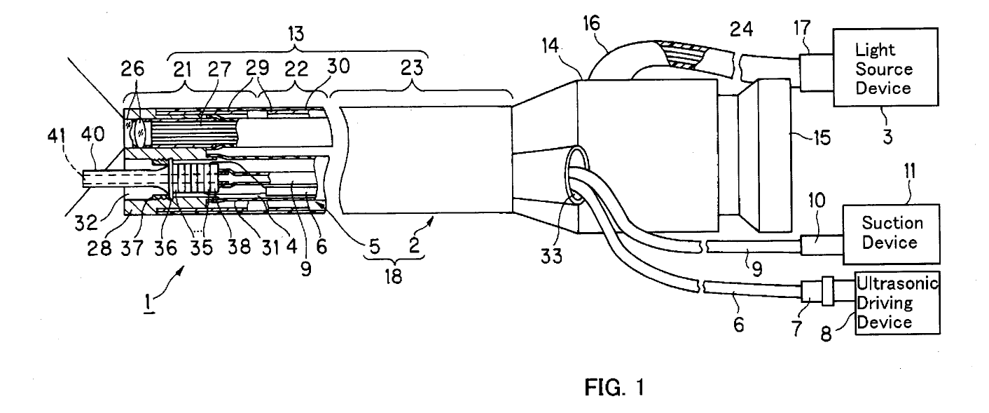

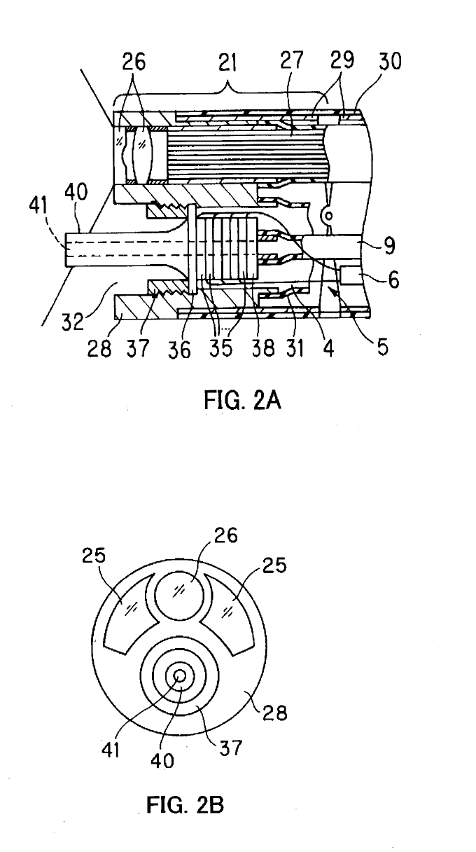

FIGS. 1, 2A and 2B are views of an ultrasonic therapeutic system and apparatus according to a first embodiment of the invention. FIG. 1 is a view showing the ultrasonic therapeutic system according to the first embodiment. FIG. 2A is an enlarged view of the distal tip portion of the ultrasonic therapeutic apparatus shown in FIG. 1, and FIG. 2B is a front view of the distal tip portion. In the first embodiment, an ultrasonic probe is fixed in a channel in the distal tip portion of an endoscope. Accordingly, a user does not need to perform the process of inserting the ultrasonic probe into the endoscope as a preparatory process before an operation nor as a process during an operation.

The ultrasonic therapeutic system 1 shown in FIG. 1 has the function of performing treatment using ultrasonic waves under endoscopic observation. The ultrasonic therapeutic system 1 includes an endoscope (body) 2 for enabling endoscopic observation, a light source device 3 for supplying illuminating light to the endoscope 2, an ultrasonic probe unit 5 incorporated in a channel 4 of the endoscope 2, an ultrasonic driving device 8 for generating an ultrasonic driving signal to be used for generation of ultrasonic waves, and a suction device 11 for sucking and collecting a treated tissue and/or fluids.

Incidentally, as a matter of course, the ultrasonic therapeutic system 1 may also be defined as an ultrasonic therapeutic apparatus which excludes the ultrasonic driving device 8, the light source device 3, and the suction device 11.

The ultrasonic driving device 8 and the ultrasonic probe unit 5 are removably connected to each other by a flexible signal cable 6 which extends from the rear end of the ultrasonic probe unit 5 and has an electrical connector 7 at its extending end for connection to a mating connector (not shown) at the ultrasonic driving device 8. The suction device 11 and the ultrasonic probe unit 5 are removably connected to each other by a flexible tube 9 which extends from the rear end of the ultrasonic probe unit 5 and has a connecting portion 10 at its extending end for connection to a mating part (not shown) at the suction device 11.

The endoscope 2 used in the first embodiment is a flexible endoscope, and has an elongated inserting section 13 having flexibility, an enlarged-diameter operating section 14 provided at the rear end of the inserting section 13, an ocular section 15 provided at the rear end of the operating section 14, and a light guide cable 16 extended from a side portion of the operating section 14. A connecting portion 17 provided at the end of the light guide cable 16 is removably connected to the light source device 3.

The ultrasonic probe unit (ultrasonic vibration unit) 5 which is an ultrasonic therapeutic instrument for giving treatment using ultrasonic waves is incorporated in the channel 4 of the endoscope 2 in a positioned state. The ultrasonic probe unit 5 may also be useful for imaging. In this manner, the endoscope 2 and the ultrasonic probe unit 5 form an ultrasonic therapeutic apparatus body 18.

The inserting section 13 of the endoscope 2 has a rigid distal tip part 21, a bending part 22 bendably provided at the rear end of the distal tip part 21, and an elongated flexible part 23 having flexibility which extends from the rear end of the bending part 22 to the front end of the operating section 14. The bending part 22 may be articulated by a user with a bending operation knob (not shown) typically located on the operating section 14, as is known in the endoscope arts.

The light source device 3 contains a lamp (not show), and illuminating light of the lamp is supplied to a light-guide end surface (not shown) in the inside of the connecting portion 17. The illuminating light supplied to the light-guide end surface is transmitted to the distal tip part 21 of the endoscope 2 through a light guide 24 (for example, an optical fiber) in the interior of the light guide cable 16 and through a light guide (not shown) inserted through the inserting section 13.

The light guide is divided into two light guides in the interior of the inserting section 13, and the respective distal tips of the divided light guides are fixed on the insides of observing windows of the distal tip part 21. Illuminating light transmitted through each of the divided light guides exits from its distal tip end surface, and this exiting light passes through the corresponding one of illuminating optical systems 25 (refer to FIG. 2B) which are disposed at the distal tip end surface in mutually opposed relationship, and exits forwardly from the endoscope 2 in a diffused manner. This exiting light illuminates a subject such as an affected area in a body cavity.

As shown in FIG. 2B, illuminating optical systems 25 are disposed on opposite sides of a front surface 28 of the distal tip part 21 of the endoscope 2, and an objective optical system (observing optical system) 26 is disposed in the center between the illuminating optical systems 25. An image of the subject illuminated via the illuminating optical systems 25 is formed by the objective optical system 26. As shown in FIGS. 2A and 1, an image guide (for example, an optical fiber) 27 is fixed in a hole portion of the distal tip part body 28 via a connecting portion at an image-forming position of the objective optical system 26.

The image guide 27 transmits an optical image to its rear end surface. The rear end surface of the image guide 27 is fixed in the vicinity of the front end of the ocular section 15. The user observes the optical image transmitted to the rear end surface of the image guide 27, at a magnified scale via an ocular optical system (not shown) of the ocular section 15.

As shown in FIGS. 1 and 2A, the front end of a first ring-shaped bending piece 29 is fixed to the periphery of the rear end of the distal tip part body 28. A second bending piece 29 is turnably connected to the rear end of the first bending piece 29, and a third bending piece 29 is turnably connected to the rear end of the second bending piece 29. In this manner, a plurality of bending pieces 29 are turnably connected in series to form the bending part 22.

The user can bend the bending part 22 in a desired direction by operating the bending operating knob (not shown) provided in the operating section 14. Incidentally, the length of the rigid distal tip part 21 is the length from the front end of the distal tip part body 28 to the rear end of the first bending piece 29. The peripheries of the respective bending pieces 29 are covered with a covering tube 30 (for example, a rubber tube).

The channel 4 is formed by a hollow flexible tube 31 inserted through the inserting section 13. The front end of the flexible tube 31 is fixedly connected to the rear end of a hole portion 32 formed in the distal tip part body 28. The hole portion 32 serves as an exit opening of the channel 4. The rear end of the channel 4 communicates with an inserting channel opening 33 near the rear end of the inserting section 13 (or the front end of the operating section 14).

A structure by which the ultrasonic probe unit 5 is incorporated will be described below. In the first embodiment, the ultrasonic probe unit 5 is incorporated in the channel 4 provided in the inserting section 13 of the endoscope 2, to form part of the ultrasonic therapeutic apparatus body 18. Accordingly, the ultrasonic therapeutic apparatus 1 has, in addition to a general endoscopic function, the function of giving treatment using ultrasonic waves.

Specifically, an ultrasonic vibrator 35 for generating ultrasonic waves for the ultrasonic probe unit 5 is mounted in the hole portion 32 (which serves as the exit opening of the channel 4) of the distal tip part body 28 of the inserting section 13. The ultrasonic vibrator 35 is mounted on a disk-shaped flange portion 36. The flange portion 36 is accommodated in the hole portion 32, and is watertightly fixed (held) in position by being fastened by a nut 37.

The ultrasonic vibrator 35 has a construction in which, for example, a plurality of disk-shaped Langevin type vibrators are stacked. The ultrasonic vibrator 35 is disposed between the flange portion 36 and a fastening member 38 (such as a screw), and is mounted to the flange portion 36 by being fastened by the fastening member 38. The flange portion 36 to which the ultrasonic vibrator 35 is mounted is fitted in the hole portion 32 and is fixed in position by being fastened by the nut 37 having a periphery around which a thread portion is formed.

The flange portion 36 having an enlarged diameter is fitted in the inside wall of the hole portion 32 and is in abutment with a stepped surface. A thread is formed around the inside circumferential surface of the hole portion 32 on the front side of the stepped surface. The nut 37 having the periphery around which the thread portion to screw into the thread is formed is fastened to fix the flange portion 36 watertightly in position. In this manner, a structure is realized which prevents water from penetrating the ultrasonic vibrator 35 on the rear side of the flange portion 36.

A horn (probe) 40 which serves as an ultrasonic wave transmitting portion (vibration transmitting portion) is fixed at its rear end surface to the front surface of the flange portion 36 by bonding or the like. Alternatively, the horn 40 can be integrally formed with the flange portion 36.

In this manner, the flange portion 36 is at least a part of a holding member which holds the ultrasonic vibrator 35 and the horn (vibration transmitting portion) 40 at the distal tip end of the inserting section 13 of the ultrasonic therapeutic apparatus 1.

In the first embodiment, the horn (probe) 40 has a conical shape which has a cross-sectional size larger at the rear end part than at its front end part so that the amplitude of vibration is increased and transmitted to the distal tip portion of the horn (probe) 40.

An ultrasonic vibration is transmitted through the horn 40 to the distal tip portion thereof having a small area size, and the distal tip portion is brought into abutment with a therapeutic target tissue to give treatment such as ablating, cauterizing or emulsifying. In this manner, the distal tip portion of the horn 40 serves as a therapeutic section for giving treatment using ultrasonic waves.

The length from the ultrasonic vibrator 35 to the distal tip of the horn 40 is set (the length of the horn 40 is adjusted) to a quarter wavelength of an ultrasonic vibration to be used, so that the ultrasonic vibration generated by the ultrasonic vibrator 35 can vibrate the distal tip portion of the horn 40 with high efficiency. In this case, a node of vibration appears at the flange portion 36 at the rear end of the horn 40, and a loop of vibration appears at the distal tip end of the horn 40.

In the first embodiment, as shown in FIGS. 1 and 2A, the ultrasonic vibrator 35 and the like are disposed in the hole portion 32 which serves as the exit opening of the channel 4 adjacent to the objective optical system 26, so that the distal tip-end side of the horn 40 enters the observing field of view of the objective optical system 26.

The fastening member 38, the ultrasonic vibrator 35, the flange portion 36 and the horn 40 have a hollow structure, and form a suction passage 41 through which to suck unnecessary tissues and the like resulting from a treatment such as ablating, cauterizing or emulsifying with the distal tip of the horn 40. The suction passage 41 communicates with the tube 9 fixedly connected to a connecting portion of the fastening member 38. This tube 9 is inserted through the channel 4 and is extended outwardly from the inserting channel opening 33.

The signal cable 6 is connected to electrodes on the surface of the ultrasonic vibrator 35. The signal cable 6 is inserted through the channel 4 and is extended outwardly from the inserting channel opening 33. The electrical connector 7 provided at the rear end of the signal cable 6 is connected to the ultrasonic driving device 8. When a foot switch (not shown) or the like is operated, a driving signal is applied to the ultrasonic vibrator 35 from the ultrasonic driving device 8, whereby ultrasonic waves can be generated by the ultrasonic vibrator 35.

In this manner, in the first embodiment, an ultrasonic vibration system part in the ultrasonic probe unit 5 is disposed in the interior of the channel 4 in the rigid distal tip part 21 of the inserting section 13, and is incorporated in the endoscope 2 in such a manner as to be watertightly fixed in position in advance. However, the distal tip portion of the horn 40 protrudes from the distal tip part 21 so that the distal tip-end side of the horn 40 enters the observing field of view of the objective optical system 26. In this construction, the user does not need an awkward process such as the process of inserting an ultrasonic probe unit into a channel and positioning a therapeutic section so that the therapeutic section can be observed in the field of view.

In addition, according to the first embodiment, merely by slightly modifying the construction of a front end portion of a channel in a general endoscope, it is possible to realize the endoscope 2 (or the ultrasonic therapeutic apparatus body 18) in which the ultrasonic probe unit 5 according to the first embodiment is incorporated.

Namely, the ultrasonic therapeutic apparatus body 18 according to the first embodiment can be realized by providing a portion for fixing the ultrasonic vibrator 35 (in the first embodiment, the hole portion 32 having the threaded inside surface) in an exit opening of a channel of a general endoscope, and fixing the ultrasonic vibrator 35 in the portion. Contrarily, as is apparent from FIG. 1, a general endoscope provided with a channel can be obtained if the thread is omitted from the hole portion 32 which serves as the exit opening of the channel 4 in which the ultrasonic vibrator 35 and the like are fixed, and if the ultrasonic vibrator 35 and the like are removed and the tube 9 and the like are removed from the channel 4. Therefore, according to the first embodiment, the ultrasonic therapeutic apparatus body 18 having an ultrasonic therapeutic function can be easily converted from an ordinary endoscope and vice versa. Accordingly, the manufacturing cost of the ultrasonic therapeutic apparatus can be reduced.

The operation of the first embodiment will be described below. The user inserts the inserting section 13 of the endoscope 2 into a body cavity, and, while observing through the ocular section 15, disposes the distal tip end of the horn 40 on an therapeutic site to be treated with ultrasonic waves. Then, the user operates operating means (not shown) for generating ultrasonic waves (for example, a foot switch or a hand switch), to apply a driving signal to the ultrasonic vibrator 35 from the ultrasonic driving device 8. Accordingly, the driving signal is converted to a mechanical vibration by the ultrasonic vibrator 35, and the thus-obtained ultrasonic vibration is transmitted to the distal tip-end side of the horn 40. In this manner, the user gives treatment such as ablation, cauterization, emulsification or the like to a living tissue by means of ultrasonic vibration under endoscopic observation.

In addition, the user can suck and collect ablated/cauterized/emulsified unnecessary tissues by activating the suction device 11.

The first embodiment has many advantages, including the following. According to the first embodiment, since the ultrasonic probe unit 5 is previously incorporated in the endoscope 2, the user does not need to perform the process of incorporating an ultrasonic probe unit before an operation or the like. Accordingly, the user does not need to perform the processes required in the related art, such as the process of disposing an ultrasonic probe so that the distal tip end thereof faces the inside of the observing field of view of an endoscope, and the complicated process of inserting an ultrasonic probe into a channel.

Accordingly, since it is not necessary to perform the process of inserting an elongated ultrasonic probe or the like into a channel before and/or during an operation, it is possible to ease stress which an operator and/or one responsible for preparation of equipment and materials may experience during the process.

Particularly in the case of a flexible endoscope such as the endoscope 2 according to the first embodiment, it is not necessary to perform the process of inserting a flexible ultrasonic probe, unlike the related art that needs such a difficult process.

In the case of the flexible endoscope according to the first embodiment, since the ultrasonic vibration system is disposed in the interior of the rigid distal tip part 21, even if an ultrasonic vibration system is made to generate a vibration when a portion following the bending part 22 is bent, stresses can be prevented from being applied to the ultrasonic vibration system, whereby it is possible to improve the durability thereof. Therefore, it is possible to improve the reliability of the ultrasonic vibration system. In addition, the ultrasonic therapeutic apparatus has a structure which makes it possible to easily incorporate the ultrasonic probe unit 5 merely by making a small modification to a flexible endoscope provided with a general channel, whereby it is possible to reduce the manufacturing cost of the ultrasonic therapeutic apparatus.

In addition, repair and maintenance of the ultrasonic probe unit 5 are facilitated. Namely, the ultrasonic probe unit 5 can be comparatively easily removed by removing the nut 37. Accordingly, repair and maintenance of the ultrasonic probe unit 5 can be easily performed. Therefore, it is possible to reduce the costs of repair and maintenance. In addition, the ultrasonic therapeutic apparatus according to the first embodiment can be used as a general endoscope if the ultrasonic probe unit 5, the signal cable 6 and the tube 9 are removed.

Incidentally, in the first embodiment, the flange portion 36 of the ultrasonic vibrator 35 is fixedly screwed into the hole portion 32 of the distal tip part body 28, but this fixing method is not to be construed as limitative. For example, a vibrator mounting frame (not shown) having a threaded inside circumferential surface may be fixed to the inside of the hole portion 32 by bonding or the like. Incidentally, the first embodiment has been described above with reference to the flexible endoscope having a construction in which the inserting section 13 is flexible, but can also be applied to a rigid endoscope having a rigid inserting section.

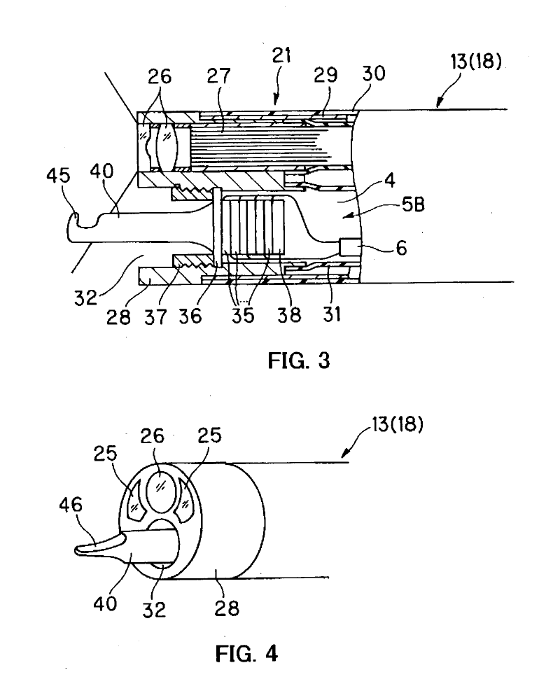

A second embodiment of the invention will be described below with reference to FIG. 3. According to the second embodiment, in the ultrasonic therapeutic apparatus according to the first embodiment, the ultrasonic vibrator 35 is made solid (no sucking space is provided), and a hook-shaped portion is formed in the distal tip portion of the horn 40.

FIG. 3 shows the structure of the distal tip-end side of the ultrasonic therapeutic apparatus body 18 according to the second embodiment. In the second embodiment, an ultrasonic probe unit 5B is used in place of the ultrasonic probe unit 5 of the first embodiment.

As shown in FIG. 3, in the ultrasonic probe unit 5B, the ultrasonic vibrator 35 and the like are solid (no sucking space is provided), and a hook-shaped portion is formed in the distal tip portion of the horn 40. In addition, the ultrasonic therapeutic apparatus according to the second embodiment has a structure in which the suction passage 41 is not formed, because the ultrasonic vibrator 35 is solid. The other constituent elements are the same in structure as the corresponding ones of the first embodiment. Therefore, in FIG. 3, the same reference numerals are used to denote the same constituent elements as those described previously in connection with the first embodiment, and the description of the same constituent elements is herein omitted.

The operation of the second embodiment will be described below. According to the second embodiment, the user can perform treatment such as coagulation or incision of a tissue by using ultrasonic waves with a hook-shaped portion 45 hooked on the tissue at a therapeutic site.

Incidentally, the distal tip portion of the horn 40 may be formed in various other shapes. For example, as shown in FIG. 4, a spatula-shaped portion 46 may also be formed. Incidentally, the horns 40 shown in FIGS. 3 and 4 may also be hollow to provide sucking as described above.

The other operation of the second embodiment is the same as that of the first embodiment, except that suction is provided through the horn 40.

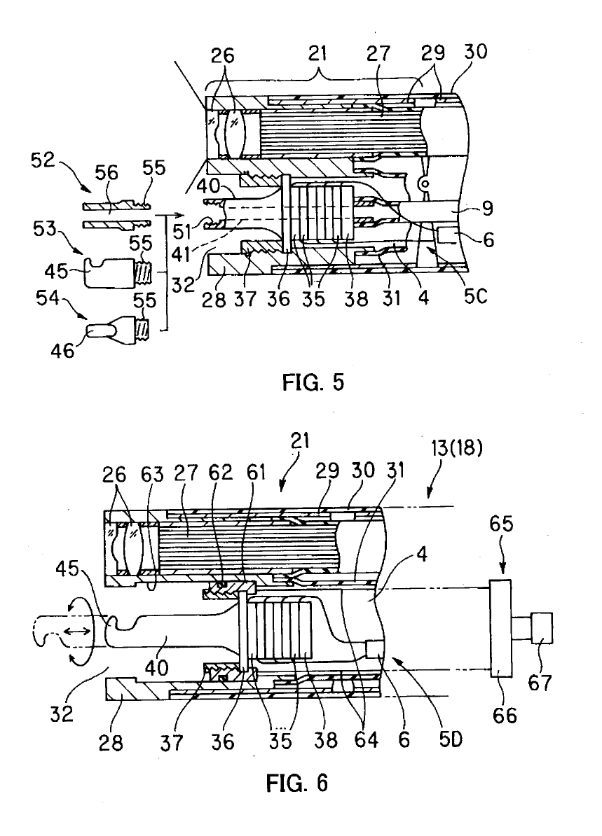

A third embodiment of the invention will be described below with reference to FIG. 5. In the ultrasonic therapeutic apparatus according to the third embodiment, the horn 40 is constructed to allow various kinds of probe members to be interchangeably attached to the distal tip portion of the horn 40 so that a probe member suitable for a particular use can be selected. FIG. 5 shows the structure of the distal tip part 21 of an ultrasonic therapeutic apparatus body according to the third embodiment. According to the third embodiment, in the ultrasonic therapeutic apparatus according to the first embodiment, the construction of part of the horn 40 provided on the front side of the ultrasonic probe unit 5 is modified into an ultrasonic probe unit 5C capable of forming an ultrasonic therapeutic system suitable for the use of each individual treatment.

The shape of the distal tip part 21 (of the endoscope 2) shown in FIG. 5 is such that the distal tip-end side of the horn (or probe) 40 is cut to shorten the horn 40 and an internal thread portion 51 is formed in the distal tip end of the shortened horn 40. Various kinds of (ultrasonic) therapeutic probe members 52, 53 and 54 suitable for individual therapeutic uses can be selectively employed in such a manner that an external thread portion 55 formed at the rear end of a selected one of the therapeutic probe members 52, 53 and 54 is fitted in the internal thread portion 51.

Each of the therapeutic probe members 52, 53 and 54 will be described below. The therapeutic probe member 52 has a hollow passage 56, and the external thread portion 55 at the rear end of the therapeutic probe member 52 can be screwed into the internal thread portion 51 to constitute an ultrasonic probe unit having a function similar to the ultrasonic probe unit 5 of the first embodiment. The therapeutic probe member 53 is solid, and has the hook-shaped portion 45 provided in its therapeutic distal tip portion as described above in connection with the second embodiment. In addition, the therapeutic probe member 54 has the spatula-shaped portion 46 provided in its therapeutic distal tip portion as shown in FIG. 4.

In the case where a selected one of these therapeutic probe members 52, 53 and 54 is fitted in the internal thread portion 51, the length from the ultrasonic vibrator 35 to the distal tip of the selected one of the therapeutic probe members 52, 53 and 54 is set to a quarter length of the wavelength of an ultrasonic vibration to be used, as described above in connection with the first embodiment. In this case, the length is set so that a node of vibration appears near the flange portion 36 and a loop of vibration appears near the distal tip end of the selected one of the therapeutic probe members 52, 53 and 54, and it is desirable that the internal thread portion 51 intermediate between the flange portion 36 and the distal tip end of the selected one of the therapeutic probe members 52, 53 and 54 be closer to the node than to the loop. In the case where the length of the rigid distal tip part 21 is large, the position of the internal thread portion 51 may be made coincident with the node of vibration by using the setting of a quarter wavelength+a half wavelength, instead of by using the above-described quarter wavelength. The other constituent elements of the second embodiment are similar in construction to the corresponding ones of the first embodiment.

The operation of the third embodiment will be described below. The user connects a desired one of the therapeutic probe members 52, 53 and 54 to the distal tip of the horn 40 according to a therapeutic use, and inserts the inserting section 13 of the endoscope 2 into a body cavity. During an operation, if a change occurs in the therapeutic use, the user draws out the endoscope 2 and replaces the selected one with another of the therapeutic probe members 52, 53 and 54. The other operation of the third embodiment is basically the same as that of the first embodiment.

The third embodiment has the following advantages in addition to the advantages of the first embodiment. Namely, it is possible to optimally select and replace the therapeutic probe members 52, 53 and 54 according to various therapeutic uses, whereby various treatments can be given by one ultrasonic therapeutic apparatus.

A fourth embodiment of the invention will be described below with reference to FIG. 6. In the fourth embodiment, the horn 40 is capable of being rotated about its central axis, and the entire horn 40 is capable of being accommodated into the endoscope 2 during insertion of the endoscope 2.

FIG. 6 shows the structure of the distal tip-end side of the ultrasonic therapeutic apparatus body 18 according to the fourth embodiment. In the fourth embodiment, an ultrasonic probe unit 5D in which the vicinity of a fixing portion of the ultrasonic vibrator 35 is modified is used in place of the ultrasonic probe unit 5B of the second embodiment.

As shown in FIG. 6, the flange portion 36 at the rear end of the horn 40 of the ultrasonic vibrator 35 is fixed to a rotating and moving member (hereinafter referred to as a rotating member) 61 which has an approximately ring-like shape and is capable of being rotated and moved, by being fastened by the nut 37 which is screwed into the rotating member 61. An O-ring 62 is fitted in a circumferential groove formed around the periphery of the rotating member 61, and a hollow portion (enlarged-diameter portion) 63 is provided in the inside circumferential surface of the hole portion 32 of the distal tip part body 28 (of the endoscope 2) so that the hollow portion 63 becomes larger in diameter than the inside circumferential surface of the hole portion 32. The O-ring 62 is engaged with the hollow portion (enlarged-diameter portion) 63 in the state of causing frictional forces to act therebetween and in the state of holding a watertight function.

The hollow portion 63 is formed to extend by a predetermined length in the axial direction. Wires 64 which serve as a transmission member for transmitting rotation and movement are connected to the rotating member 61 at their front ends, and these wires 64 pass through the channel 4 and are connected to a rotation and movement operating member 65 on the operating-section side of the endoscope 2.

The rotation and movement operating member 65 has a disk 66 to which the wires 64 are fixed at their rear ends and an operating knob 67 secured to the center shaft of the disk 66. When the operating knob 67 is rotated, the rotating member 61 can be rotated, and when the operating knob 67 is moved back and forth (in FIG. 6, rightwardly and leftwardly), the rotating member 61 can be moved back and forth in the axial direction within the hollow portion 63. The other constituent elements of the fourth embodiment are basically the same in construction as the corresponding ones of the first embodiment.

The operation of the fourth embodiment will be described below. When the user rotationally operates the rotation and movement operating member 65 provided on the operating-section side of the endoscope 2, the wires 64 are interclockingly driven and the rotating member 61 is rotationally driven against the sliding friction of the O-ring 62. As a result, the ultrasonic vibrator 35 fixed to the rotating member 61 is rotationally operated.

In addition, when the user operates the rotation and movement operating member 65 to move it back and forth, the rotating member 61 is moved back and forth in the axial direction within the range of the predetermined length of the hollow portion 63. In this manner, the ultrasonic vibrator 35 fixed to the rotating member 61 is operated to move back and forth.

The axial length of the hollow portion 63 is selected so that when the rotating member 61 is moved back to the closest position to the operating-section side, the distal tip portion of the horn 40 connected to the ultrasonic vibrator 35 is accommodated into the hole portion 32 which serves as the exit opening of the channel 4. This state is shown by solid lines in FIG. 6.

When the user is to insert or draw out the distal tip part 21 of the endoscope 2 into or from a body cavity, the distal tip portion of the horn 40 connected to the ultrasonic vibrator 35 is set to the state of being accommodated in the hole portion 32 which serves as the exit opening of the channel 4 as shown by two dot-dash lines in FIG. 6. When the user is to perform treatment using ultrasonic waves, the user protrudes the therapeutic section (in this case, the hook-shaped portion 45) provided in the distal tip portion of the horn 40, from the hole portion 32 as shown by the solid lines in FIG. 6, and places the distal tip portion into the observing field of view and performs treatment using ultrasonic waves, as by rotating the distal tip portion to an angle suitable for incision or the like.

The other operation is the same as those of the second embodiment.

The fourth embodiment has the following advantages in addition to the advantages of the first and second embodiments. Namely, since the distal tip portion of the horn 40 of the ultrasonic vibrator 35 can be rotationally driven, it is possible to realize a far more accurate approach to a therapeutic section. Accordingly, an operator can easily give treatments, and can perform high-quality treatments. In addition, in the case where the ultrasonic therapeutic apparatus is used in combination with a flexible endoscope, the advantage of the fourth embodiment can be doubled when the fourth embodiment is used in combination with the bending operation of the flexible endoscope.

In addition, since the horn 40 can be slid in the axial direction to retract the distal tip portion of the horn 40 into the channel 4 when no treatment is being performed, the inserting section 13 of the endoscope 2 can be made to approach a therapeutic section far more easily during the insertion of the inserting section 13 into a body cavity, whereby during the insertion, the horn 40 of the ultrasonic therapeutic apparatus does not enter the observing field of view so that the user can ensure a clear observing field of view.

Incidentally, in the fourth embodiment, the distal tip portion of the horn (or probe) 40 has the hook-shaped portion 45, but the shape of the distal tip portion of the horn 40 is not limited to only a hook-like shape. For example, the spatula-shaped portion 46 may be used, and a construction in which various therapeutic probe members having different kinds of distal tip shapes can be removably attached as described above in the third embodiment may also be used.

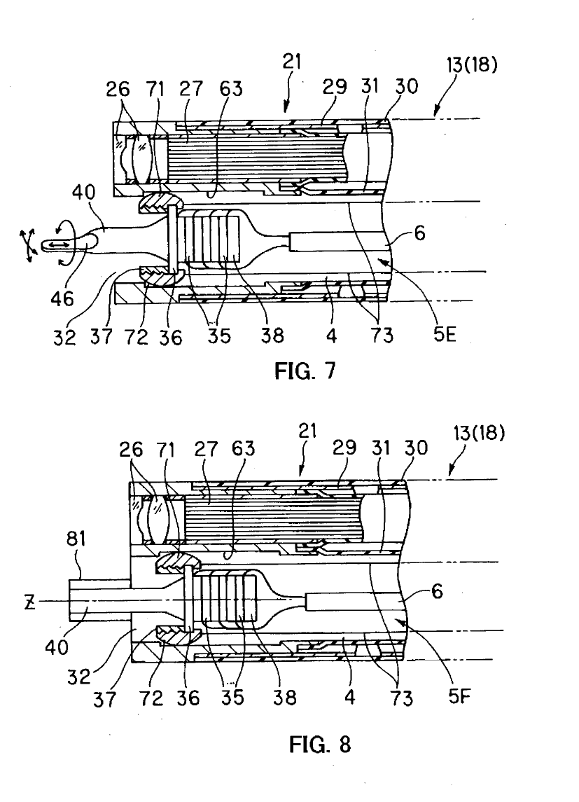

A fifth embodiment of the invention will be described below with reference to FIG. 7. The fifth embodiment has the feature that the horn 40 can be tilted, in addition to the feature of the fourth embodiment.

FIG. 7 shows the structure of the distal tip-end side of the ultrasonic therapeutic apparatus body 18 according to the fifth embodiment. In the fifth embodiment, an ultrasonic probe unit 5E which is a modification of the ultrasonic probe unit 5D of the fourth embodiment is used in place of the ultrasonic probe unit 5D. The ultrasonic probe unit 5E basically uses a joint member 71 capable of tilting more than the rotating member 61 shown in FIG. 6, and is constructed to enable tilting operations to be performed on the operating-section side.

As shown in FIG. 7, the flange portion 36 to which the ultrasonic vibrator 35 is fixedly connected is fixed to the joint member 71. The joint member 71 has an approximately ring-like shape, and its peripheral surface is formed as a spherical portion 72. This spherical portion 72 is watertightly engaged with the hollow portion 63 provided in the hole portion 32 which forms the exit opening 32 of the channel 4.

The hollow portion 63 is formed to extend by a predetermined length in the axial direction. Wires 73 (for example, four wires) which also effect joint driving are connected to the joint member 71, and these wires 73 pass through the channel 4 and are connected to a joint operating section (not shown) on the operating-section side of the endoscope 2. The joint operating section includes means for tiltably supporting the disk 66 in the rotation and movement operating member 65 shown in FIG. 6, and enables the user to perform the operation of tilting the operating knob 67. The user can tilt the joint member 71 in various directions, such as upward, downward, rightward and leftward, by performing the operation of tilting the operating knob 67.

The axial length of the hollow portion 63 is selected so that when the rotating member 71 is moved back to the closest position to the operating-section side, the distal tip portion of the horn 40 connected to the ultrasonic vibrator 35 is accommodated into the channel 4. The other constituent elements are the same in structure as the corresponding ones of the first embodiment.

The operation of the fifth embodiment will be described below. When the user operates the joint operating section to perform the operation of alternately pulling and pushing the wires 73 which also effect joint driving, the joint member 71 is driven to tilt through the operation of the spherical portion 72 of the joint member 71. In this manner, the flange portion 36 and the ultrasonic vibrator 35 which are fixed to the joint member 71 are tilted. Accordingly, the distal tip portion of the horn 40 connected at the rear end to the flange portion 36 is tilted as indicated by the crossed arrows in FIG. 7.

The other operation is the same as that of the fourth embodiment. As described above in connection with the fourth embodiment in particular, the hollow portion 63 has the predetermined axial length, and when the user operates joint operating means to move the joint member 71, the joint member 71 is moved back and forth in the axial direction within the range of the hollow portion 63, whereby the ultrasonic vibrator 35 and the horn 40 are moved back and forth. Similarly, the user can rotate the ultrasonic vibrator 35 and the horn 40 by rotationally operating the joint operating means.

The fifth embodiment has the following advantage in addition to the advantages of the first and fourth embodiments. Namely, the distal tip portion of the horn 40 of the ultrasonic vibrator 35 can be driven to tilt in a larger number of arbitrary directions than those in the fourth embodiment, whereby the above-described advantage of the fourth embodiment is further improved. In addition, it is possible to realize not only ultrasonic treatment but also blunt peeling or the like using the distal tip portion of the horn 40 connected to the ultrasonic vibrator 35.

Incidentally, in the fifth embodiment, the distal tip portion of the horn 40 connected to the ultrasonic vibrator 35 has the spatula-shaped portion 46, but the shape of the distal tip portion of the horn 40 is not limited to only a spatula-like shape. For example, the hook-shaped portion 45 or the hollow shape described above in connection with the first embodiment may be used, and a construction in which various therapeutic probe members having different kinds of distal tip shapes can be removably attached as described above in the third embodiment may also be used.

A sixth embodiment of the invention will be described below with reference to FIGS. 8 to 10. The sixth embodiment has, in addition to the feature of the fifth embodiment, the feature that the distal tip part body 28 of the endoscope 2 is provided with a jaw so that a living tissue or the like can be grasped between a tiltable horn and the jaw.

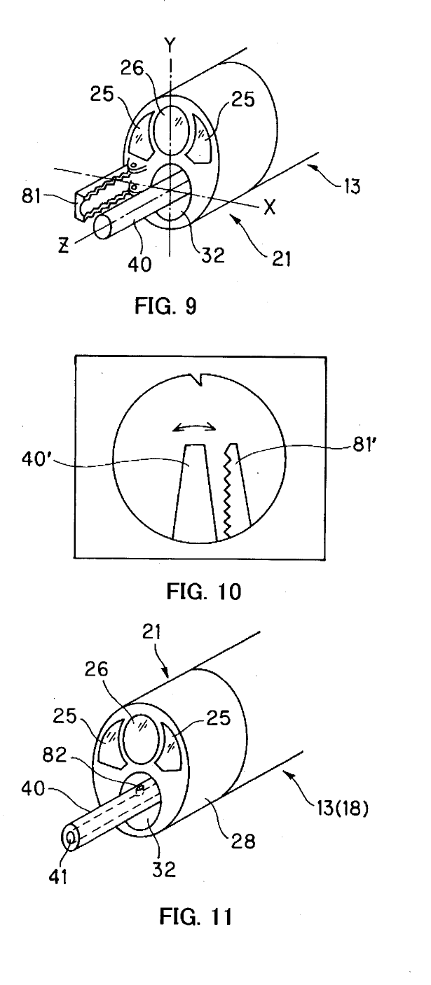

FIG. 8 shows the structure of the distal tip-end side of the ultrasonic therapeutic apparatus body 18 according to the sixth embodiment, FIG. 9 shows in perspective view the distal tip-end side shown in FIG. 8, and FIG. 10 shows an example of the observing field of view of the ultrasonic therapeutic apparatus body 18. In the sixth embodiment, an ultrasonic probe unit 5F which is a modification of the ultrasonic probe unit 5E of the fifth embodiment is used in place of the ultrasonic probe unit 5E.

The sixth embodiment has a basic construction in which, as shown in FIG. 8, a jaw 81 is provided on the distal tip part body 28 so that a living tissue or the like can be treated in the state of being clamped between the distal tip portion of the rod-shaped tiltable horn 40 and the jaw 81.

As shown in FIGS. 8 and 9, when no tilting operation is being performed, a central axis (Z) which passes through the center of the ultrasonic vibrator 35 and the center of the horn 40 connected thereto is arranged to extend along the optical axis of the objective optical system 26 (i.e., along the axis of the cylindrical distal tip part 21 of the endoscope 2).

As shown in FIG. 9, during this state, at the distal tip end surface, the center of the objective optical system 26 is arranged so that the center of the horn 40 is positioned on an axis (Y) crossing the optical axis of the horn 40 at right angles.

Furthermore, the approximately plate-shaped jaw 81 is arranged to protrude from the distal tip end surface along the central axis Z at a position spaced apart from the central axis Z of the horn 40 along an axis (X) crossing the axis Y at right angles.

A hollow portion whose shape corresponds to, for example, the shape of the distal tip-end side of the horn 40 is formed on the surface of the jaw 81 that is opposed to the horn 40, in such a manner that the hollow portion extends along the center line of the surface. Corrugated portions are respectively formed on the opposite sides of the hollow portion so that a tissue can be clamped firmly between the jaw 81 and the horn 40. This jaw 81 is formed of a low-friction and heat-resistance material such as a fluororesin (for example, Teflon®). The other constituent elements are the same in construction as the corresponding one of the fifth embodiment.

The operation of the sixth embodiment will be described below. As shown in FIG. 10, the user puts the distal tip portion of the horn 40 and the jaw 81 close to a therapeutic site to be treated, while observing an image 40′ of the distal tip portion of the horn 40 and an image 81′ of the jaw 81. Then, the user clamps a tissue of the therapeutic site between the distal tip portion of the horn 40 and the jaw 81.

In this case, similarly to the case of the fifth embodiment, the joint member 71 is tilted like a joint by the operation of the joint operating means, and the distal tip portion of the horn 40 is tilted toward the jaw 81 to grasp the tissue between the distal tip portion of the horn 40 and the jaw 81.

Then, the user performs the operation of generating ultrasonic waves, to generate frictional heat due to vibration so that the grasped tissue can be coagulated and/or incised.

In this manner, the sixth embodiment serves, in addition to the advantages of the above-described first to fifth embodiments, the advantage that the user can perform treatments such as coagulation and incision on a therapeutic tissue far more reliably and easily, by clamping the therapeutic tissue.

A seventh embodiment of the invention will be described below with reference to FIG. 11. In the seventh embodiment, small holes are provided in the peripheral wall of the horn 40 of the ultrasonic therapeutic apparatus according to the first embodiment, thereby enabling collection of a mist.

FIG. 11 shows the structure of the distal tip-end side of the ultrasonic therapeutic apparatus body 18 according to the seventh embodiment. The seventh embodiment provides an example in which the ultrasonic vibrator 35 of the first embodiment is improved.

As shown in FIG. 11, the horn 40 connected to the ultrasonic vibrator 35 has at least one small hole 82 on the distal tip side from the flange portion 36, and the at least one small hole 82 communicates with the hollow suction passage 41. The at least one small hole 82 is disposed at a position opposed to the objective optical system 26. The other constituent elements are the same in construction as the corresponding ones of the first embodiment.

The operation of the seventh embodiment will be described below. During ultrasonic treatment, in the case where moisture neighboring a tissue is scattered as mist, the mist is sucked and collected through the small hole 82.

The seventh embodiment serves the following advantage in addition to the advantage of the first embodiment. Namely, since mist can be collected, a far better field of view can be secured.

In the case where the seventh embodiment is combined with cleaning means for the objective optical system 26 of the endoscope 2, it is possible to automatically suck and collect a cleaning liquid, whereby it is not only possible to secure a far better field of view, but it is also possible to omit a suction operation and improve the operability of an operator. Furthermore, since it is possible to suck and circulate air surrounding the objective optical system 26, it is possible to prevent a fog from occurring on the objective optical system 26. Incidentally, the invention further encompasses other embodiments which are formed by combining part of the above-described embodiments.

As is apparent from the foregoing description, according to each of the first to seventh embodiments, since an ultrasonic vibration is contained in an endoscope, it is possible to omit the process of positioning an ultrasonic therapeutic instrument at the distal tip end of the endoscope and the process of inserting the ultrasonic therapeutic instrument through a channel.

Furthermore, since the process of positioning an observing optical system in an observing field of view is not needed, the difficult and trouble some process of inserting the ultrasonic therapeutic instrument can be eliminated to enable an operator and the like to avoid stress due to such an inserting process. In addition, when the ultrasonic vibrator is combined with a flexible endoscope, no stress is applied to an ultrasonic vibration system, whereby it is possible to realize an ultrasonic therapeutic apparatus which is highly durable and hence highly reliable.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. An ultrasonic therapeutic apparatus comprising:
an endoscope having an inserting section to be inserted into an object;
an ultrasonic vibrator for generating ultrasonic vibration;
a vibration transmitting section for transmitting the ultrasonic vibration generated by the ultrasonic vibrator to the object; and
a holding member for holding the ultrasonic vibrator and the vibration transmitting section in a distal tip portion of the inserting section of the endoscope.

2. An ultrasonic therapeutic apparatus according to claim 1, wherein the vibration transmitting section is an ultrasonic probe coupled to the ultrasonic vibrator in such a manner as to be capable of transmitting the ultrasonic vibration generated by the ultrasonic vibrator, and wherein the holding member holding the ultrasonic probe so that a distal tip portion of the ultrasonic probe protrudes from the inserting section of the endoscope at least during treatment.

3. An ultrasonic therapeutic apparatus according to claim 2, wherein the ultrasonic probe has a therapeutic section for giving treatment to the object.

4. An ultrasonic therapeutic apparatus according to claim 2, wherein the distal tip portion of the ultrasonic probe that protrudes from the inserting section of the endoscope includes a therapeutic section for giving treatment to the object.

5. An ultrasonic therapeutic apparatus according to claim 1, wherein the holding member is positioned at a node of ultrasonic vibration generated on the basis of a vibration of the ultrasonic vibrator.

6. An ultrasonic therapeutic apparatus according to claim 1, wherein the vibration transmitting section has a length equivalent to a quarter wavelength of the ultrasonic wave.

7. An ultrasonic therapeutic apparatus according to claim 1, wherein the vibration transmitting section has a hollow passage.

8. An ultrasonic therapeutic apparatus according to claim 1, wherein the vibration transmitting section is solid.

9. An ultrasonic therapeutic apparatus according to claim 1, wherein the vibration transmitting section is solid and has a hook-shaped distal tip portion.

10. An ultrasonic therapeutic apparatus according to claim 1, wherein the vibration transmitting section is solid and has a spatula-shaped distal tip portion.

11. An ultrasonic therapeutic apparatus according to claim 1, wherein the ultrasonic vibrator and the vibration transmitting section are included in an ultrasonic vibration unit, and wherein the ultrasonic vibration unit being held in a hole portion communicating with a channel provided in the distal tip portion of the inserting section of the endoscope.

12. An ultrasonic therapeutic apparatus according to claim 11, wherein the ultrasonic vibration unit is connected to a flexible cable inserted through the channel.

13. An ultrasonic therapeutic apparatus according to claim 11, wherein the ultrasonic vibration unit is connected to a flexible tube inserted through the channel.

14. An ultrasonic therapeutic apparatus according to claim 1, wherein the inserting section of the endoscope is rigid at the distal tip portion and has a flexible portion except the distal tip portion.

15. An ultrasonic therapeutic apparatus according to claim 1, wherein the ultrasonic vibrator and the vibration transmitting section are included in an ultrasonic vibration unit, and wherein the holding member fixes the ultrasonic vibration unit in position in a hole portion communicating with a channel of the endoscope.

16. An ultrasonic therapeutic apparatus according to claim 2, wherein the ultrasonic probe is removably attached to the ultrasonic vibrator.

17. An ultrasonic therapeutic apparatus according to claim 16, wherein a location at which to removably attach the ultrasonic probe corresponds to a node of vibration.

18. An ultrasonic therapeutic apparatus according to claim 2, wherein the ultrasonic probe has a length equivalent to a quarter wavelength of the ultrasonic wave

19. An ultrasonic therapeutic apparatus according to claim 2, wherein the ultrasonic probe is hollow and the ultrasonic vibrator is also hollow.

20. An ultrasonic therapeutic apparatus according to claim 2, wherein the ultrasonic probe is made of a solid member having a hook-shaped tip portion.

21. An ultrasonic therapeutic apparatus according to claim 2, wherein the ultrasonic probe is made of a solid member having a spatula-shaped tip portion.

22. An ultrasonic therapeutic apparatus according to claim 2, wherein the ultrasonic vibrator is fixed to a rotary member rotatable with respect to a hole portion provided in the distal tip portion of the inserting section, the rotary member being capable of being rotationally operated by an operating member connected to the rotary member via a transmission member.

23. An ultrasonic therapeutic apparatus according to claim 2, wherein the ultrasonic vibrator is fixed to a joint member movable like a joint with respect to a hole portion provided in the distal tip portion of the inserting section, the joint member being capable of being tilted by an operating member connected to the joint member via a transmission member.

24. An ultrasonic therapeutic apparatus according to claim 2, wherein the ultrasonic probe is movable in an axial direction of a hole portion provided in the distal tip portion of the inserting section.

25. An ultrasonic therapeutic apparatus according to claim 2, wherein the ultrasonic probe is movable within at least a range in which when the ultrasonic probe is moved to a distal-most position, the distal tip portion of the ultrasonic probe is positioned in an observing field of view, while when the ultrasonic probe is moved to a proximal-most position, the distal tip portion of the ultrasonic probe is stored in the hole portion.

26. An ultrasonic therapeutic apparatus according to claim 2, further comprising a jaw, the jaw being disposed at an end surface of the distal tip portion of the inserting section at a position where a line connecting the ultrasonic probe and the jaw crosses at right angles a line connecting the ultrasonic probe and an observing optical system.

27. An ultrasonic therapeutic apparatus according to claim 2, wherein the ultrasonic probe has a hollow passage extending in an axial direction thereof, and further has at least one small hole communicating with the hollow passage, on a front side from a position where the ultrasonic vibrator is fixed.

28. An ultrasonic therapeutic apparatus for giving treatment under endoscopic observation, comprising:
an endoscope having a channel in its inside;
an ultrasonic vibrator which is at least partly accommodated in the channel and converts an electrical signal into a mechanical vibration; and
an ultrasonic probe coupled to the ultrasonic vibrator with at least a distal tip portion of the ultrasonic probe positioned in an observing field of view of the endoscope, the ultrasonic probe transmitting an ultrasonic vibration generated by the ultrasonic vibrator.

29. An ultrasonic therapeutic apparatus according to claim 28, wherein the ultrasonic probe has a hollow passage.

30. An ultrasonic therapeutic apparatus according to claim 28, wherein the ultrasonic probe is solid.

31. An ultrasonic therapeutic apparatus according to claim 28, wherein the ultrasonic probe has a hook-shaped distal tip portion.

32. An ultrasonic therapeutic apparatus according to claim 28, wherein the ultrasonic probe has a spatula-shaped distal tip portion.

33. An ultrasonic therapeutic apparatus according to claim 28, wherein the ultrasonic probe is removably attached to the ultrasonic vibrator, a location at which to removably attach the ultrasonic probe corresponding to a node of vibration.

34. An ultrasonic therapeutic apparatus according to claim 28, wherein the ultrasonic probe has a length equivalent to a quarter wavelength of an ultrasonic wave

35. An ultrasonic therapeutic apparatus according to claim 28, wherein the ultrasonic probe and the ultrasonic vibrator are hollow.

36. An ultrasonic therapeutic apparatus according to claim 28, wherein the ultrasonic probe is a solid member having a hook-shaped distal tip portion.

37. An ultrasonic therapeutic apparatus according to claim 28, wherein the ultrasonic probe is a solid member having a spatula-shaped distal tip portion.

38. An ultrasonic therapeutic apparatus according to claim 28, wherein the ultrasonic vibrator is fixed to a rotary member rotatable with respect to the channel of the endoscope, the ultrasonic therapeutic apparatus further comprising a transmitting member connected to the rotary member and an operating member connected to the transmitting member.

39. An ultrasonic therapeutic apparatus according to claim 28, wherein the ultrasonic vibrator is fixed to a joint member movable with respect to the channel of the endoscope for tilting the ultrasonic probe, the ultrasonic therapeutic apparatus further comprising a transmitting member connected to the joint member and an operating member connected to the transmitting member.

40. An ultrasonic therapeutic apparatus according to claim 28, wherein the ultrasonic probe is movable in the channel of the endoscope in an axial direction of the channel.

41. An ultrasonic therapeutic apparatus according to claim 28, wherein the ultrasonic probe is movable within at least a range in which when the ultrasonic probe is moved to a distal-most position, the distal tip portion of the ultrasonic probe is positioned in an observing field of view, while when the ultrasonic probe is moved to a proximal-most position, the tip portion of the ultrasonic probe is stored in the channel.

42. An ultrasonic therapeutic apparatus according to claim 39, further comprising a jaw, the jaw being disposed at an end surface of a distal tip portion the inserting section of at a position where a line connecting the ultrasonic probe and the jaw crosses at right angles a line connecting the ultrasonic probe and an observing optical system.

43. An ultrasonic therapeutic apparatus according to claim 28, wherein the ultrasonic probe has a hollow passage extending in an axial direction thereof, and further has at least one small hole communicating with the hollow passage, on a front side from a position where the ultrasonic vibrator is fixed in the channel of the endoscope.

44. An ultrasonic therapeutic apparatus comprising:
an endoscope;
an ultrasonic vibrator held in a tip portion of the endoscope in such a manner as not to be removed by at least a user; and
an ultrasonic probe to which a vibration of the ultrasonic vibrator is transmitted.

45. An ultrasonic therapeutic apparatus according to claim 44, wherein a distal tip portion of the ultrasonic probe is positioned in an observing field of view of the endoscope.

46. An ultrasonic therapeutic apparatus according to claim 44, wherein the endoscope has a channel, the ultrasonic vibrator being at least partly accommodated in the channel.

47. An ultrasonic therapeutic apparatus according to claim 46, further comprising a mechanism for moving the ultrasonic probe back and forth according to an operation of the user.

48. An ultrasonic therapeutic apparatus according to claim 47, wherein when the ultrasonic probe is moved to a distal-most position, a distal tip portion of the ultrasonic probe is positioned distally to a front end of the channel, while when the ultrasonic probe is moved to a proximal-most position, the distal tip portion of the ultrasonic probe is positioned proximally to the front end of the channel.

49. An ultrasonic therapeutic apparatus according to claim 47, wherein when the ultrasonic probe is moved to the distal-most position, a distal tip portion of the ultrasonic probe is positioned in an observing field of view of the ultrasonic probe.

50. An ultrasonic therapeutic apparatus according to claim 44, further comprising a mechanism for rotating the ultrasonic probe according to an operation of the user.

51. An ultrasonic therapeutic apparatus according to claim 44, further comprising a mechanism for tilting the ultrasonic probe according to an operation of the user.

52. An ultrasonic therapeutic apparatus according to claim 51, further comprising a jaw for grasping an object in cooperation with the ultrasonic probe, the jaw being provided in the distal tip portion of the endoscope.

53. An ultrasonic therapeutic apparatus according to claim 52, wherein a distal tip portion of the jaw is positioned in an observing field of view of the endoscope.

54. An ultrasonic therapeutic apparatus according to claim 46, wherein the ultrasonic vibrator is positioned entirely in the channel, and wherein the ultrasonic therapeutic apparatus further comprising a member for sealing the ultrasonic vibrator in the channel.

55. An ultrasonic therapeutic apparatus according to claim 54, wherein the member for sealing also has a function to hold the ultrasonic vibrator.

56. An ultrasonic therapeutic apparatus according to claim 44, wherein the ultrasonic vibrator is mounted with a screw.

57. An ultrasonic therapeutic apparatus according to claim 44, wherein the ultrasonic probe is interchangeably removable from the ultrasonic vibrator.

57. An ultrasonic therapeutic apparatus according to claim 44, wherein the ultrasonic probe has a suction passage in its inside.

58. An endoscope comprising a channel having a structure for holding at least one of an ultrasonic vibrator and an ultrasonic probe, the structure being formed in an inside wall of a distal tip end opening of the endoscope.

59. An endoscope according to claim 58, wherein the structure is a thread formed on the inside wall.

60. An endoscope comprising:
an ultrasonic probe positioned in a distal portion of the endoscope;
means for transmitting ultrasonic vibration to the ultrasonic probe; and
means for one of fixing and movably fixing the ultrasonic probe to the distal portion.
